(19) United States
(12) Patent Application Publication
(10) Pub. No.: US 2022/0001151 A1
(43) Pub. Date: Jan. 6, 2022

(54) METHOD OF MANUFACTURING BALLOON CATHETER AND BALLOON CATHETER MANUFACTURED THEREFROM

(71) Applicant: Besmed Health Business Corporation, New Taipei City (TW)

(72) Inventors: Tao-Tsun HSIUNG, New Taipei City (TW); Kuan-Hung CHEN, Chiayi County (TW); Wei HSIUNG, New Taipei City (TW)

(73) Assignee: Besmed Health Business Corporation, New Taipei City (TW)

(21) Appl. No.: 17/014,381

(22) Filed: Sep. 8, 2020

(30) Foreign Application Priority Data
Jul. 3, 2020 (TW) 109122558

(51) Int. Cl. A61M 25/10; B29C 48/11; B29C 48/12; B29C 48/23

(52) U.S. Cl. CPC A61M 25/1029; B29C 48/11; B29L 2031/7543; B29C 48/23; A61M 2025/1031; B29C 48/12

(57) ABSTRACT

A method of fabricating a balloon catheter configuration is provided, the method comprises: preparing one catheter body, the catheter body comprises one internal tube, one external tube, and one stripping agent; the internal tube comprises one longitudinal groove and one drain passageway, wherein the longitudinal groove is configured in the exterior surface of the internal tube, the drain passageway is configured inside the internal tube, the external tube covers the internal tube, and the stripping agent is configured between the internal tube and the external tube; removing the internal tube of the catheter to allow one end of the external tube of the catheter body beyond one end of the internal tube adjacent to the external tube; folding the end of the external tube toward the axial center of the catheter; and attaching the end of the external tube to one part of the inner wall of the drain passageway.

### CROSS-REFERENCE TO RELATED APPLICATIONS

The present application is a non-provisional application which claims the benefit of priority of Taiwanese Patent Application No. 109122558, filed on Jul. 3, 2020.

### TECHNICAL FIELD

The present disclosure relates to a method of fabricating a balloon catheter, in particular, to provide a simplified method associated with the fabrication process for a balloon catheter. In addition, the present disclosure relates to a balloon catheter configuration in accordance with the method of the fabrication process therefrom.

### BACKGROUND

In the process of certain medical treatments, users have to insert a balloon catheter into a living body in order to expand a lumen or create a space inside the living body to be treated, or increase the function of a support force to improve the efficiency in the process of a medical operation. Moreover, it provides the convenience and flexibility for medical users to transmit a nutrient or medicine into the living body by applying the balloon catheter so as to secure a nutrient supply or operate a treatment in the living body.

Referring to FIG. 10, the fabrication process of a conventional balloon catheter in accordance with a prior art comprising the certain steps is as illustrated below:

(1) A silicone-based material is applied in an extruder to form a conduit 90 by using a heating and an extrusion process;

(2) Drilling and forming hole configuration on the side wall close to one end of the conduit 90 to form hole configuration 91 by penetrating through the sidewall of the conduit 90, and the hole configuration 91 is configured in the section A of the conduit 90;

(3) In the conduit 90, a peeling layer 92 is formed on the inner and outer walls of section A; and

(4) Place another silicone-based material at positions P1 , P2, and P3 of section A, and, by using a glue attachment, make the silicone-based material and the interior surface as well as the exterior surface of the conduit 90 are glued three times or more, thereby forming a balloon structure on the conduit 90.

From this perspective, it is obvious that the fabrication process of the balloon catheter in accordance with the prior art has to perform certain steps of drilling holes on the side surface of the conduit 90 as well as at least three times necessary associated with attachment procedures, so that the balloon structure can be formed on the conduit 90.

Therefore, the fabrication method of the balloon catheter in accordance with conventional technology has to go through sophisticated procedures associated with the fabrication process of the balloon catheter, resulting in the increase of time consumption in the manufacturing process. Furthermore, the use of multiple adhesions leads to excessive residual concentration associated with the frequently use of glue and increase the risk of contamination in the medical environment and the manufacturing cost.

In view of the foregoing, a method of fabricating a balloon catheter is provided in order to minimize fabrication steps efficiently and reduce manufacturing time consumption, and a balloon catheter configuration is fabricated in accordance with the method therefrom.

### SUMMARY

The present disclosure preferably provides a fabrication method of a balloon catheter and additionally, a balloon catheter is formed in accordance with the method hereof. The balloon catheter is configured and applied in the clinical applications such as a urinary catheter, a hematuria catheter, an endotracheal tube, a gastrostomy tube, unique catheters in vivo for pyeloplasty, nephrostomy, tubal reshaping, and etc. The main part of the balloon catheter comprises an internal tube having a tubular wall, and a longitudinal groove configured on the tubular wall of the internal tube incorporating a drain passageway. The longitudinal groove of the internal tube is covered by an overcoat sheath. In a catheter body of the balloon catheter, wherein, in one part, the stripping agent is configured between the overcoat sheath and the tubular wall of the internal tube, the overcoat sheath and the tubular wall are not combined, and in other part, no stripping agent is applied, the overcoat sheath and the tubular wall of the internal tube are combined mutually. Throughout of the time underlying certain steps in the fabrication process, the stripping agent preferably volatilizes through the channels coupled to the external ambience, and the degree of volatilization (totally or partially) differs in accordance with the types of stripping agents applied in the fabrication process. Therefore, in the present disclosure, if the balloon catheter is applied to insert into or pull out of the human body, the pain and injury of the patient can be further alleviated. In addition, the present disclosure is configured according to an integral assembly process and in this way, avoids the accumulation of waste fluids (eg. urine) inside the body effectively, due to the input distance gap between the balloon configuration and the drain passageway is further minimized, thereby it eliminates effectively the waste fluid that will be possibly retained inside the body (eg. bladder). Furthermore, it can be applied to nutrient delivery systems (eg. gastrostomy tube) efficiently, in the case of penetrating deeply into the human tissue, the distal end part of balloon catheter, incorporating the balloon configuration, can be appropriately sustained between the tissue and the opening so as to improve and facilitate the function of an internal securement module inside the human body in process of the medical application.

The present disclosure provides a method of fabricating a balloon catheter, by forming a balloon catheter with an external tube covering the internal tube and forming a groove on the exterior surface of the internal tube, the process steps of the balloon catheter fabrication can be reduced efficiently. In addition, the time consumption of manufacturing the balloon catheter is minimized, and the process cost of the balloon catheter is eligible to be reduced accordingly. Furthermore, as a result of the complication of the procedures and the requirement of mass production in the fabrication process, it may cause more process variations in the process of fabricating the balloon catheter, and increase the risk such as the shrinkage rate of a product, sacrifice the accuracy of the product specification, and so on, resulting in poor yields, lack of stability and reliability, which may cause medical personnel clinical issues and health hazards to patient. For the avoidance of the above issues, the mentioned advantages can be alleviated in accordance with reducing the fabrication steps of the balloon catheter.

Another embodiment of the present disclosure provides a balloon catheter configuration in accordance with the method of fabricating the balloon catheter, apart from this, it facilitates medical users in the medical application and improves the efficacy and efficiency associated with medical operations.

For the purpose of achieving the above objectives, the present disclosure provides a method applied to fabricate a balloon catheter in accordance with one preferred embodiment, which comprises certain steps as described below: a silicone-based material is applied into an extruder to form a catheter body, and the catheter body preferably comprises an internal tube, an external tube, and a stripping agent; the internal tube preferably comprises a longitudinal groove and a drain passageway; wherein the longitudinal groove, along the axial direction of the internal tube, is preferably configured on the exterior surface of the internal tube, and extending from one end of the internal tube to the other end of the internal tube; and wherein the drain passageway, along the axial direction of the internal tube, is preferably configured inside the internal tube and extending from one end of the internal tube to the other end of the internal tube; covering the internal tube by the external tube; the stripping agent is preferably configured on the distal part of the catheter body and additionally, is preferably configured between the internal tube and the external tube; wherein one partial interior surface of the external tube configured on the distal part of the catheter body is separated from one partial exterior surface of the internal tube adjacent to the external tube, and thereby a working space can be formed and preferably configured above the exterior surface of the internal tube; within the working space, in the axial direction of the catheter body, a procedure of removing one part of the internal tube configured in the catheter body is applied so that one end of the external tube of the catheter body exceeds one end of the internal tube, which is adjacent to the external tube; wherein the working space defines a space located availably above the part of the outer wall of the internal tube and is sufficient for the user to apply the procedure of removing the part of the internal tube flexibly ; and after the procedure of removing the part of the internal tube is completed, the end of the external tube configured in the catheter body, toward the axial center and the inner radial direction of the catheter body, is preferably folded in the interior surface of the drain passageway configured in the internal tube, and connected with the inner wall of the drain passageway of the internal tube by using an adhesion procedure, so as to configure a balloon configuration to be inflated.

In one embodiment, a silicone-based material is applied in an extruder to configure and form a catheter body, the catheter body comprises an internal tube, an external tube, and a stripping agent; the internal tube comprises a longitudinal groove, and a drain passageway, wherein the longitudinal groove, along the axial direction of the internal tube, is configured on the outer wall of the internal tube, and extending from one end of internal tube until the other end of internal tube; and wherein the drain passageway, along the axial direction of the internal tube, is configured inside the internal tube, and extending from one end of the internal tube until the other end of the internal tube; the external tube covers the internal tube; the stripping agent, configured on the distal part of the catheter body, is formed between the internal tube and the external tube; wherein one end of the external tube configured on the distal part of the catheter body faces away from the axial center of the catheter body (i.e. axially outwardly) and toward the outer radial direction; fold axially outwardly so as to expose partially the internal tube of the catheter body and followed by applying a procedure of removing the part of the internal tube; and after the procedure of removing the part of the internal tube is completed, the end of the external tube is preferably unfolded (i.e. restored or released) toward the axial direction of the catheter body so that, along the axial direction, the end of the external tube of the catheter body extends beyond the end of the internal tube adjacent to the external tube; and toward the axial center and the inner radial direction of the catheter body, fold the end of the external tube configured at the first end part of the catheter body axially inwardly, toward the axial center of the catheter body, into the drain passageway of the internal tube. Afterwards, the folded part is attached to one part of the inner wall of the drain passageway configured in the internal tube.

In one embodiment, a silicone-based material is applied in an extruder to process and form a catheter body, the catheter body comprises an internal tube, an external tube, and a stripping agent; the internal tube comprises a longitudinal groove, and a drainage pipe, wherein the longitudinal groove, along the axial direction of the internal tube, is configured on the outer wall of the internal tube, and extends from one end of the internal tube until the other end of the internal tube; wherein the drain passageway, along the axial direction of the internal tube, is configured inside the internal tube and extends from one end of the internal tube until the other end of the internal tube; the external tube covers the internal tube; and the stripping agent, configured on the distal part of the catheter body, is formed in one part of the interface between the internal tube and the external tube; wherein the drain passageway comprises a separation structure, extending along the axial direction of the internal tube, configured connectedly to the inner wall of the internal tube, and the volume (e.g. lumen area) associated with the drain passageway is partitioned into portions configured inside the internal tube so as to provide the function of flow shunting in use.

In one embodiment, the separation structure preferably comprises: a sheet-plate structure, a triangular-pyramid structure, a polygonal structure, a herringbone structure, an H-shaped structure, a cross structure, or a combination of the above structures.

In one embodiment, one end of the separation structure is preferably in undercut configuration (i.e retracted axially inwardly into the internal tube), wherein the longitudinal length of the tubular wall of the internal tube in the axial direction is greater than the longitudinal length of the separation structure in the axial direction.

In one embodiment, the inwardly folded part is preferably in adhesion to the inner wall of the drain passageway configured inside the internal tube. The adhesion process herein can be applied preferably by using an adhesive, heat welding treatment, and pressure treatment, ultrasonic bonding or enhancement process, or a combination of the above methods.

In one embodiment, the inwardly folded part is preferably in adhesion to the inner wall of the drain passageway configured inside the internal tube. Prior to the adhesion process is applied, a procedure of surface treatment is further applied on one part of the inner wall of the drain passageway and the procedure of the surface treatment process comprises: performing surface roughening, applying heating, or adding an intermediate layer.

In one embodiment, a process of surface modification is further applied on one part of the inner wall of the drain passageway. In addition, the process of surface modification is applied to roughen the part of the inner wall of the drain passageway. The surface of the inner wall is preferably configured as a concave-convex surface, a spiral surface, or other alternative approaches so as to increase the surface area of the inner wall.

In one embodiment, the concave-convex surface comprises a primary concave-convex surface and a secondary concave-convex surface configured on at least one part of the primary concave-convex surface.

In one embodiment, the method of fabricating the balloon catheter further comprises steps: an infusion connector is configured at the other end of the catheter body opposite to the first end part of the catheter body; wherein the infusion connector comprises an air inlet, and a feeding pipe; the air inlet and the longitudinal groove of the internal are in communication mutually; in addition, the feeding pipe is coupled to the drain passageway of the internal tube, and the air inlet and the feeding pipe are coupled not in communication mutually.

In one embodiment, in the step of applying the silicone-based material into the extruder for the fabrication, wherein the diameters, extending from one end of the catheter body to the other end, are preferably equivalent.

In one embodiment, in the step of applying the silicone-based material into the extruder for fabrication, the diameter of the internal tube and the diameter of the external tube of the catheter body are respectively reduced toward the distal end of the catheter body, wherein one part at vicinity of the distal end of the catheter body is arranged to form a balloon configuration to be inflated.

In one embodiment, in the step of applying the silicone-based material into the extruder for fabrication, the diameter of the internal tube and the diameter of the external tube of the catheter body are respectively increased toward the distal end of the catheter body, wherein one part at vicinity of the distal end of the catheter body is arranged to form a balloon configuration to be inflated

In one embodiment, in the step of applying the silicone-based material into the extruder for fabrication, the catheter body further comprises an X-ray indicator, and the X-ray indicator is configured at one location relative to a dent configuration of the internal tube, wherein the dent configuration is a cross-sectional structure illustrated in accordance with the cross section of the longitudinal groove of the internal tube in a perspective view.

In addition, the present disclosure provides a balloon catheter in accordance with the method of fabricating the balloon catheter, comprising: a catheter body, the catheter body comprising: an internal tube comprises a longitudinal groove and a drain passageway; wherein the longitudinal groove is configured on the outer wall of the internal tube, along the axial direction of the internal tube, and extends from the proximal end of the internal tube to the distal end of the internal tube; and wherein the drain passageway is configured inside the internal tube, along the axial direction of the internal tube, and extends from the proximal end of the internal tube to the distal end of the internal tube; an external tube covers the internal tube; and a stripping agent, which is configured in the distal end of the catheter body in the specific step of the fabrication process (certain step previously), is configured between the internal tube and the external tube; wherein the longitudinal length of the external tube in the first end part of the catheter body is longer than the longitudinal length of the internal tube and the end of the external tube configured in the first end part of the catheter body is folded inwardly to the internal tube. In addition, the end of the external tube configured in the first end part of the catheter body is preferably folded inwardly to one part of the inner wall of the internal tube and adheres to each other.

In one embodiment, the balloon catheter further comprises an infusion connector configured at the proximal end of the catheter body, wherein the distal end of the catheter body is preferably arranged to configure with the balloon configuration to be inflated. In addition, the infusion connector comprises an air inlet and a feeding pipe; wherein the air inlet communicates with the longitudinal groove of the internal tube; the air inlet communicates with the drain passageway of the internal tube; and the air inlet and the feeding pipe are not in communication with each other.

In one embodiment, the infusion connector comprises an air inlet and at least one feeding pipe; wherein the air inlet is coupled to the longitudinal groove configured on the internal tube; wherein the at least one feeding pipe and the drain passageway of the internal tube are configured in communication mutually, and the air inlet and the at least one feeding pipe are not configured in communication with each other.

In one embodiment, the diameter within the catheter body, extending from one end until the other end, is equivalent equal.

In one embodiment, the diameter of the internal tube and the diameter of the external tube of the catheter body are respectively reduced toward the distal end of the catheter body, wherein the distal end of the catheter body is arranged to configure with the balloon configuration to be inflated, thereby preventing a round-shape module is configured at this end, additionally, as such that the patient's pain and injury can be reduced and the fabrication process is simplified when the balloon catheter is inserted or pulled out.

In one embodiment, the diameter of the internal tube and the external tube of the catheter body increase respectively toward the end of the catheter body, wherein the end of the catheter body is arranged to configure with the balloon configuration to be inflated. In the perspective of the increase in the inner diameter and outer diameter of the end part of the catheter body, when folded into the drain passageway of the internal tube, the end of the external tube configured at the first end part of the catheter body faces toward the axial center of the catheter body as well as in the inward radial direction, and the inwardly folded part is then attached to the inner wall of the drain passageway of the internal tube. Consequently, in this regard, the wrinkles or crumples possibly formed on the inwardly folded part of the external tube can be further prevented due to the increase associated with the inner diameter of the end of the catheter body as well as the mismatch of surface area for a implementation of an appropriate attachment, thereby the effect of a stable adhesion can be achieved in accordance with a simplified adhesion procedure hereof.

In one embodiment, in the step of applying the silicone-based material into the extruder for fabrication, the catheter body further comprises an X-ray indicator, and the X-ray indicator is configured at a position relative to the longitudinal groove.

In one embodiment, the catheter body further comprises an X-ray indicator, and the X-ray indicator is configured at a position relative to a dent configuration of the internal tube by looking into in terms of a cross section of the catheter body.

According to the present disclosure, in the fabrication process of the balloon catheter, one adhesion step is required to configure the balloon configuration of the balloon catheter. In addition, through the silicone-based material is applied in the extruder for fabrication, the internal tube incorporating the longitudinal groove is configured, and in this regard, additional steps underlying drilling the internal tube is avoidable. In this way, the method of fabricating the balloon catheter in accordance with the present disclosure not merely offers simplified steps, but effectively reduces manufacturing costs and reduces time consumption as well. Furthermore, the balloon catheter described in the present disclosure is preferably applied to penetrate into living organisms with small openings, and the X-ray indicator of the balloon catheter allows users to identify the dent easily and promptly. Therefore, the balloon catheter disclosed in the present disclosure enables to improve the efficiency in the process of the medical operation.

According to the description of the present disclosure and objectives, purpose, features, configures, effects, and advantages that can be more obvious and easy to understand, the present disclosure will cite specifically preferred embodiments, in conjunction with the drawings and descriptions in detail as follows. In addition, the directional terms described in the present disclosure, such as up, down, bottom, front, back, left, right, inside, outside, tubular wall, sidewall, inner wall, outer wall, center, longitudinal, axial, axis, axial center, radial, inner radial, outer radial, inward radial, outward radial, toward, or away from are for reference in the direction of the attached drawings. Therefore, the directional terms applied are not intended to limit the scope of the present disclosure.

The following preferred embodiments allow the present disclosure to be more comprehensively understood by one of ordinary skill in the art, but do not limit the spirit, coverage, and/or scope of the present disclosure in any way whatsoever. The aspects of the present disclosure and the details thereof are illustrated more fully by referring to the non-limiting embodiments and examples disclosed in conjunction with the following description and the accompanying drawings. Descriptions on known information, manufacturing tools, process technologies, and etc. are omitted, so as not to avoid obscure the present disclosure. However, it should be understood that the following description and preferred embodiments, which are provided to illustrate the aspects or concepts of the present disclosure, are by means of indicating illustration and not limitation. One of ordinary skill in the art will be able to recognize that various embodiments of the invention and numerous specific details thereof, many substitutions, alterations, modifications, additions, and/or arrangements may be configured within the spirit and/or scope of the inventive concept through the present disclosure without departing from the spirit thereof, and embodiments of the invention include all such substitutions, modifications, additions and/or rearrangements.

### BRIEF DESCRIPTION OF THE DRAWINGS

FIG. 1 is a flow chart showing certain steps associated with the fabrication process of providing a balloon catheter in accordance with the first embodiment of the present disclosure.

FIG. 2 is a top view of the first balloon catheter configured in accordance with procedure S1 associated with the method of fabricating a balloon catheter in the first embodiment of the present disclosure.

FIG. 3 is a cross-sectional view of the first balloon catheter configured in accordance with procedure 51 associated with the method of fabricating a balloon catheter in the first embodiment of the present disclosure.

FIG. 4 is a top view of the balloon catheter configured in accordance with procedure S2 associated with the method of fabricating a balloon catheter in the first embodiment of the present disclosure.

FIG. 5A is a top view of the balloon catheter to be inflated and configured in accordance with procedure S3 associated with the method of fabricating a balloon catheter in the first embodiment of the present disclosure.

FIG. 5B is a top view of another balloon catheter to be inflated and configured in procedure S3 associated with the method of fabricating a balloon catheter of the first embodiment of the present disclosure.

FIG. 6 is a perspective view of a balloon catheter configured by applying the method of fabricating a balloon catheter in accordance with the first embodiment of the present disclosure.

FIG. 7A is a top view of a balloon catheter configured by applying the method of fabricating a balloon catheter in accordance with the first embodiment of the present disclosure after the balloon configuration in balloon catheter is inflated.

FIG. 7B is a top view of another balloon catheter configured by applying the method of fabricating a balloon catheter in accordance with the first embodiment of the present disclosure after the balloon configuration in balloon catheter is inflated.

FIG. 8 is a top view of a balloon catheter configured by applying the method of fabricating a balloon catheter in accordance with the second embodiment of the present disclosure.

FIG. 9 is a top view of a balloon catheter configured by applying the method of fabricating a balloon catheter in accordance with the third embodiment of the present disclosure.

FIG. 10 shows a balloon catheter fabricated in accordance with a conventional prior art.

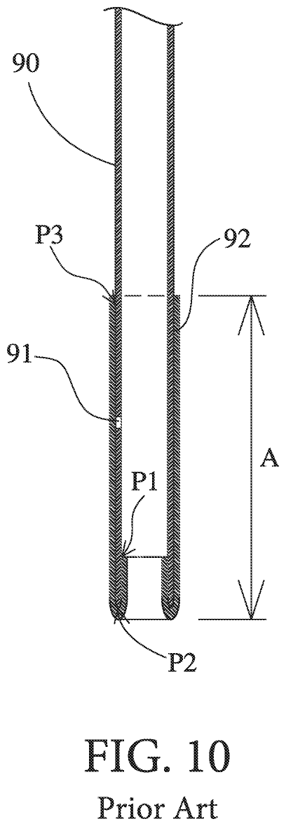

The implementations disclosed herein are illustrated by way of example, and not by way of limitation, in the figures of the accompanying drawings. Like reference numerals refer to corresponding parts throughout the drawings.

### DETAILED DESCRIPTION

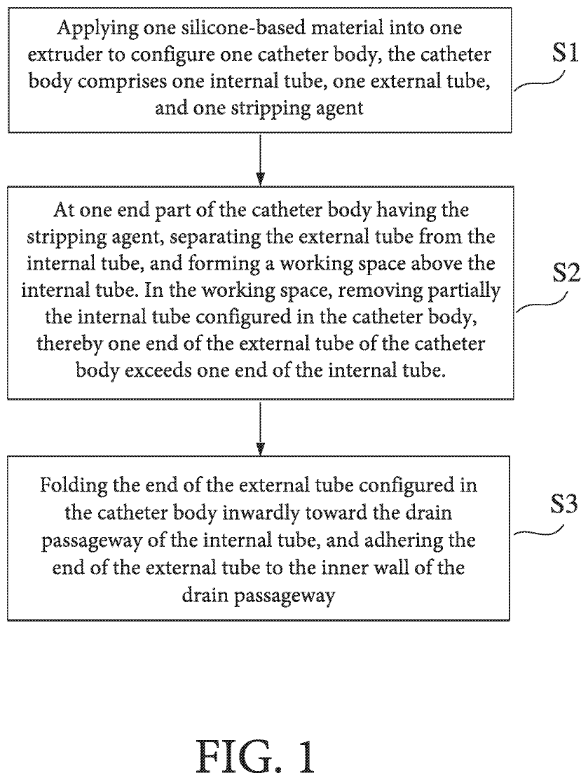

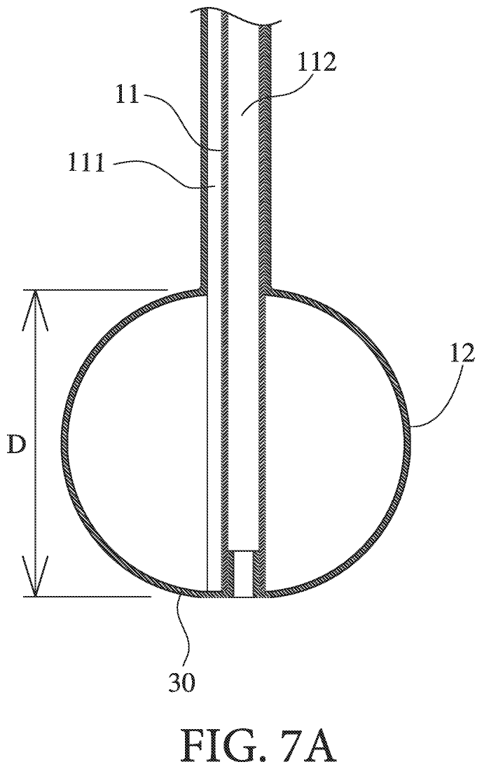

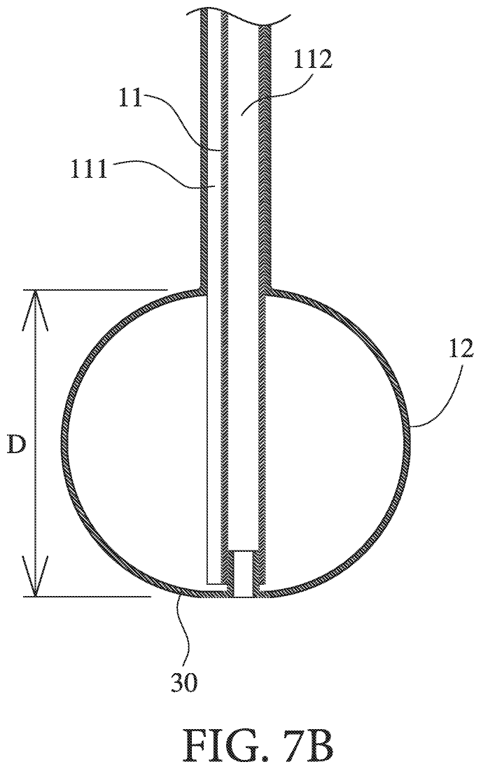

Referring to FIGS. 1 to 7A, and 7B, the fabrication method of the balloon catheter is provided in accordance with the first embodiment of the present disclosure comprises steps:

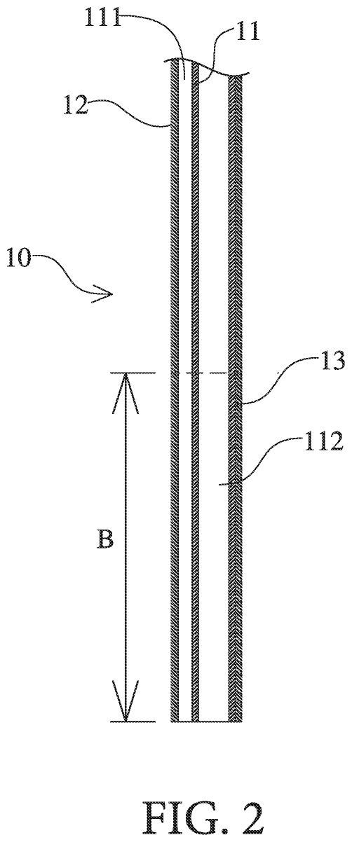

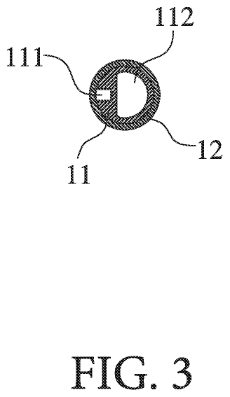

Step S1: Apply a silicone-based material into an extruder for fabrication to form a catheter body 10. By referring to FIGS. 2 and 3, the catheter body 10 comprises an internal tube 11, an external tube 12 and a stripping agent 13; and the internal tube 11 comprises a longitudinal groove 111 and a drain passageway 112, wherein the longitudinal groove 111 is configured on the exterior surface of the internal tube 11, and, along the axial direction of the internal tube 11, extending from one end of the internal tube 11 to the other end of the internal tube 11; and wherein the drain passageway 112 is configured inside the internal tube 11, along the axial direction of the internal tube 11, and extends from one end of the internal tube 11 to the other end of the internal tube 11; in addition, the drain passageway 112 is configured in parallel with the longitudinal groove 111 and not in communication with each other, wherein the external tube 12 covers the internal tube 11 and the tubular length of the external tube 12 is equivalent to the tubular length of the internal tube 11. Furthermore, the stripping agent 13, configured in the section B of the catheter body 10, is arranged in one interface part between the internal tube 11 and the external tube 12, and volatilizes in the process of step 51, wherein the section B is configured in the first part of the catheter body 10.

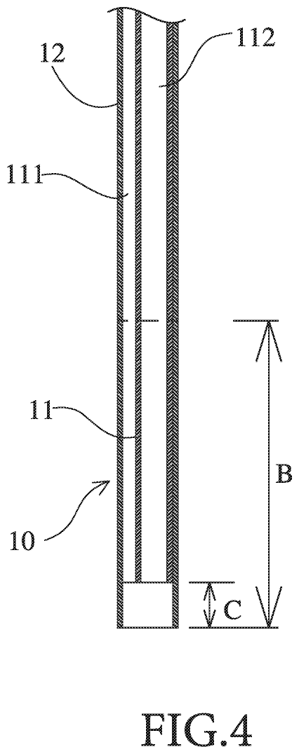

Step S2: Referring to FIG. 4, one end of the external tube 12 in the section B of the catheter body 10 is preferably separated from the internal tube 11 and thus a working space is formed and preferably configured above the internal tube 11 in the section B. In the working space, in the axial direction, the internal tube 11 in the section C configured of the catheter body 10 is partially (or fully) removed (i.e. one part of the internal tube is removed), and a cross-sectional surface of the internal tube is formed, consequently, in this regard, in the axial direction, the end of the external tube 12 of the catheter body 10 exceeds the end of the internal tube 11, which is adjacent to the external tube 12; that is, in the region of the catheter body configured close to the section C of the catheter body 10, the length of the external tube 12 of the catheter body 10 is longer than the length of the internal tube 11 of the catheter body 10.

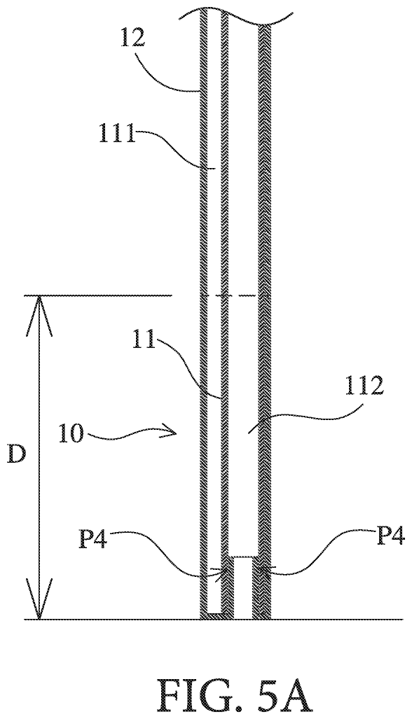

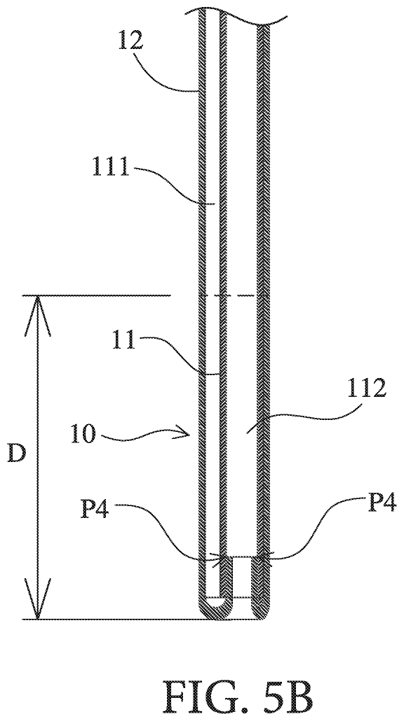

Step S3: Referring to FIGS. 4, 5A, and 5B, upon completion of a procedure of removing the internal tube partially, the end of the external tube 12 in the section C configured in the catheter body 10 is moved toward the axial center of the catheter body 10 and folded inwardly toward the inner radial direction of the drain passageway 112 of the internal tube 11; wherein the position P4 as well as the interior surface of the drain passageway 112 of the internal tube 11 are incorporated to form a balloon configuration to be inflated in accordance with using an adhesion procedure. The adhesion procedure is applied preferably by using an adhesive glue, a heat welding, a pressure treatment, an ultrasonic bonding process, or a combination of the above procedures: wherein the position P4 is configured at vicinity of the end part of the catheter body 10 and one part of the interior surface of the drain passageway 112.

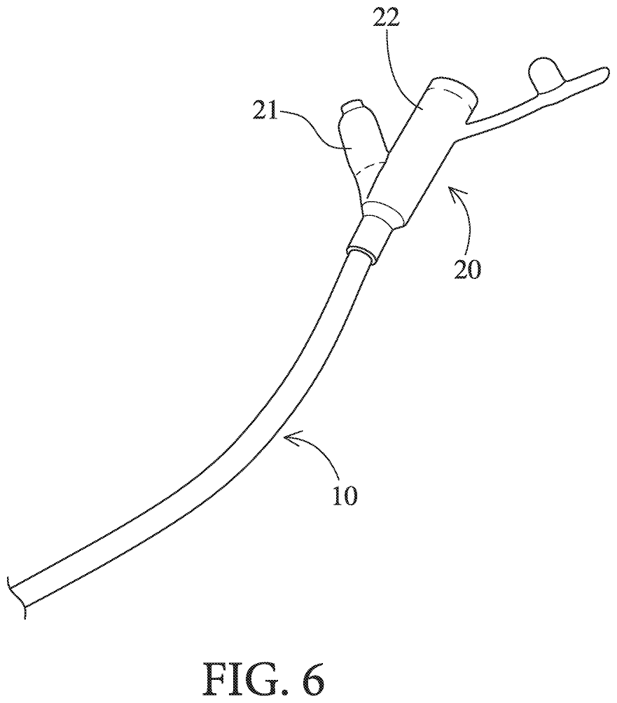

Referring to FIGS. 5A, 5B, and 6, in the process of clinical procedure, relative to the end of the catheter body 10, users preferably configures an infusion connector 20 coupled to the other end of the catheter body 10, relative to the end of the catheter body 10 where is arranged to configure the balloon configuration to be inflated; wherein the infusion connector 20 comprises an air inlet 21 and a feeding pipe 22, and the air inlet 21 communicates with the longitudinal groove 111 of the internal tube 11 mutually; in addition, the feeding pipe 22 communicates with the drain passageway 112 of the internal tube 11; and the air inlet 21 and the feeding pipe 22 are not coupled in communication with each other.

Referring to FIGS. 5A, 6, and 7A, in the process of a medical procedure, when applying a balloon catheter configured in accordance with the method of fabricating a balloon catheter in the first embodiment of the present disclosure, first of all, the balloon configuration to be inflated is preferably configured at one end of the catheter body 10 and applied in a living body. In addition, a syringe is coupled to the air inlet 21 of the infusion connector 20, and air is injected into the balloon configuration to be inflated through the syringe by supplying the air through the longitudinal groove 111 of the internal tube 11 and entering the end of the catheter body 10, wherein the end of the catheter body 10 is arranged to equip with the balloon configuration to be inflated, so that the external tube 12 configured in the section D of the catheter body 10 expands away from the internal tube 11 and an inflated balloon configuration 30 is formed accordingly. Through the function of the inflated balloon configuration 30 provided, the space required in the living body can be expanded, thereby facilitating and improving medical operation efficiency. Furthermore, it is applied to inject a nutrient or a medication into the balloon catheter by coupling an injector to the feeding pipe 22 of the infusion connector 20, so that the nutrient or the medication passes through the internal tube 11. The drain passageway 112 is used and applied to enter the living body and supplies nutrition or medicine into a living body.

Referring to FIGS. 5B, 6, and 7B, when a balloon catheter configured in accordance with another fabrication method of the first embodiment in the present disclosure is applied in a medical procedure, first of all, one part of one end of the catheter body 10 incorporating the balloon configuration to be inflated is preferably applied into a living body, and a syringe is coupled to the air inlet 21 of the infusion connector 20, and air is injected into the balloon catheter through the syringe to pass the air through the longitudinal groove 111 of the internal tube 11 until the part of the end of the catheter body 10, where is delicately arranged to equip with the balloon configuration to be inflated, so that the part of the external tube 12 located in the section D of the catheter body 10 expands toward the outer radial direction of the internal tube 11. To form an inflated balloon configuration 30 in the section D, additionally, the external tube 12 configured adjacent to the end of the internal tube 11 and the balloon configuration to be inflated is further expended, including the expansion in the axial and radial direction of the internal tube 11 (i.e including the expansion at the end terminal of the internal tube 11).

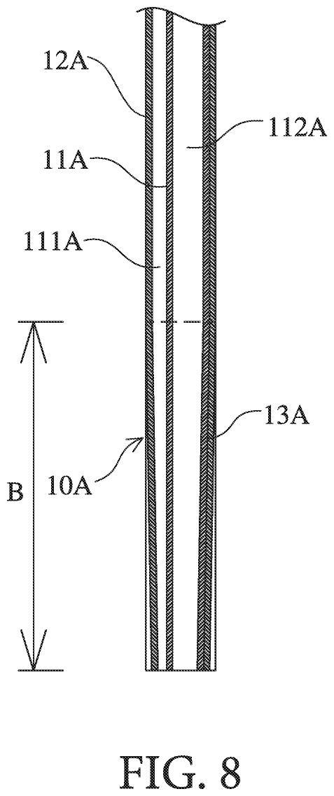

Referring to FIG. 8, the fabrication method of the balloon catheter provided by the second embodiment of the present disclosure is provided, comprising; a catheter body 10A comprises an internal tube 11, an external tube 12, and a stripping agent 13A to be formed, wherein the internal tube 11 comprises a longitudinal groove 111A and a drain passageway 112A. As compared to the first embodiment of the present disclosure, the main differences between the second embodiment of the present disclosure and the first embodiment include: The catheter body 10A by step S1 forms a cone-shaped structure, toward the end of the catheter body 10A, at one end of the catheter body 10A to which the balloon configuration to be inflated is to be formed. That is, the diameter of the internal tube 11A and the diameter of the external tube 12A of the catheter body 10A respectively decrease or increase toward the end of the catheter body 10A, where the balloon configuration to be inflated is to be arranged (i.e. one part of section B). As shown in FIG. 8, the balloon catheter fabricated in accordance with the method for providing a balloon catheter in accordance with the second embodiment of the present disclosure is configured by forming a cone-shaped structure at one end part of the catheter body 10A, where is arranged to equip with the balloon configuration to be inflated. The balloon catheter can be easily and smoothly penetrated into a living body through an entrance having a small opening, or reduce the pain and injury of the patient when the catheter is inserted, removed, or pulled out, and thereby improve the efficiency of medical operation.

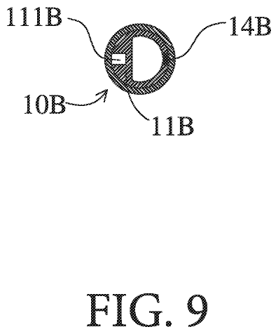

Referring to FIG. 9, the fabrication process of the balloon catheter provided by the third embodiment of the present disclosure is provided by referencing the first embodiment of the present application. The differences between the third embodiment and the first embodiment of the present disclosure are mainly described as below: The catheter body 10B configured in step Si further comprises an X-ray indicator 14B, wherein the X-ray indicator 14B is provided opposite to a dent 111B in term of a cross sectional view, configured in the internal tube 11B. The balloon catheter fabricated in accordance with the method of manufacturing a balloon catheter in the third embodiment of the present disclosure enables the users to identify promptly the position of the dent 111B according to the X-ray indicator 14B, thereby improves the medical operation efficiency.

In summary, as compared to the prior art, the fabrication process of the balloon catheter applied in the present disclosure, one adhesion step is required to form a balloon configuration in the balloon catheter. In addition, as shown in FIG. 2, the fabrication process of the balloon catheter in the present disclosure is applied to configure the internal tube 11 incorporating the longitudinal groove 111 in the procedure of step Si, and therefore additional efforts associated with drilling hole in the internal tube 11 is avoidable. Therefore, the fabrication process of the balloon catheter in the present disclosure not merely provides a simplified process, but effectively reduces the manufacturing cost and minimize the production time as well. Furthermore, the balloon catheter fabricated in accordance with the method of configuring a balloon catheter in the present disclosure can be efficiently penetrated into a living body through a small opening and entrance, and the X-ray indicator of the balloon catheter is applied to be promptly identified by users, respective to the position or orientation of the longitudinal groove, in this way, the balloon catheter configured in the present disclosure allows to improve the medical operation efficiently.

Although the present disclosure has been discussed in the above embodiments, they are not intended to limit the scope of the present disclosure. Any one skill in the art can make changes and embellishment without departing from the spirit and scope of the present disclosure. Therefore, the scope of the present disclosure shall be accorded to the definitions set forth in the appended claims.

What is claimed is:

1. A method for fabricating one balloon catheter, the method comprising the following steps:
applying one silicone-based material into one extruder to configure one catheter body, the catheter body comprises one internal tube, one external tube, and one stripping agent;
wherein the internal tube comprises one longitudinal groove and one drain passageway,
the longitudinal groove, along the axial direction of the internal tube, is configured on the exterior surface of the internal tube, and extending from one end of the internal tube to the other end of the internal tube; and
the drain passageway, along the axial direction of the internal tube, is configured inside the internal tube, and extending from one end of the internal tube to the other end of the internal tube;
wherein the external tube covers the internal tube; and
wherein the stripping agent, configured at the first end part of the catheter body, is applied between one part of the exterior surface of the internal tube and one part of the interior surface of the external tube;
at the first end part of the catheter body, separating the part of the interior surface of the external tube configured from the part of the exterior surface of the internal tube;
forming one working space above the part of the exterior surface of the internal tube;
wherein, in the working space, applying one procedure of removing one part of the internal tube so that one end of the external tube at the first end part of the catheter body exceeds one end of the internal tube;
after the procedure of removing the part of the internal tube is completed, toward the axial center of the internal tube, folding the end of the external tube beyond the end of the internal tube inwardly into the drain passageway of the internal tube; and
adhering the end of the external tube to one part of the inner wall of the drain passageway, so as to configure one balloon configuration to be inflated.

2. The method according to claim 1, wherein the method of separating the part of the interior surface of the external tube configured at the first end part of the catheter body from the part of the exterior surface of the internal tube, the method comprising steps of:
at the first end part of the catheter body, away from the axial center of the catheter body,
folding the end of the external tube to expose one part of the internal tube of the catheter body; and
applying the procedure of removing the part of the internal tube configured in the catheter body.

3. The method according to claim 1, wherein the method of adhering the end of the external tube to the part of the inner wall of the drain passageway, the method comprising the use of applying adhesive attachment, heat welding treatment, pressure treatment, ultrasonic bonding process, or a combination of thereof.

4. The method according to claim 3, further comprising steps of:
applying one surface treatment on the part of the inner wall of the drain passageway, wherein the surface treatment method comprises of roughening, heating, applying one intermediary layer, or a combination of thereof.

5. The surface treatment method according to claim 4, prior to applying the surface treatment on the part of the interior surface of the drain passageway, wherein the surface treatment further comprises the procedures of
configuring one concave-convex surface, one spiral surface, a combination of thereof, or other approach applied to increase the surface area of the interior surface of the drain passageway.

6. The method according to claim 1, the method further comprising steps of:
configuring one infusion connector at the other end of the catheter body relative to the first end part of the catheter body, where is arranged to configure the balloon configuration to be inflated;
wherein the infusion connector comprises one air inlet and one feeding pipe, the air inlet and the longitudinal groove configured in the internal tube are in communication mutually; and the drain passageway of the feeding pipe and the internal tube are in communication mutually and the air inlet and the feeding pipe are not in communication with each other.

7. The method according to claim 1, wherein, in the step of applying the silicone-based material into the extruder to configure the catheter body, the diameter of the internal tube and the diameter of the external tube are configured in the catheter body and are reduced respectively toward the first end part of the catheter body.

8. The method according to claim 1, wherein, in the step of applying the silicone-based material into the extruder to configure the catheter body, the diameter of the internal tube and the diameter of the external tube are configured in the catheter body and are increased respectively toward the first end part of the catheter body.

9. The method according to claim 1, further comprising steps of configuring one separation structure in the drain passageway of the internal tube and the separation structure is configured connectedly to the interior surface of the drain passageway, wherein the separation structure extends in the axial direction of the internal tube and shunts the flow volume configured inside the drain passageway of the internal tube.

10. The method according to claim 9, further comprising step of retracting one end of the separation structure into the internal tube, wherein the length of the internal tube in the axial direction is greater than the length of the separation structure in the axial direction.

11. A balloon catheter configuration, the balloon catheter configuration comprising:
one catheter body, the catheter body comprising:
one internal tube incorporating one longitudinal groove and one drain passageway;
wherein the longitudinal groove, along the axial direction of the internal tube, is configured on the exterior surface of the internal tube, and extending from one end of the internal tube to the other end of the internal tube; and
wherein the drain passageway, along the axial direction of the internal tube, is configured inside the internal tube, and extending from one end of the internal tube to the other end of the internal tube;
one external tube covering the internal tube; and
one balloon configuration to be inflated is configured at the first end part of the catheter body, and the balloon configuration to be inflated comprises one part of the internal tube and one part of the external tube;
wherein, in the outer radial direction of the internal tube, and the part of the internal tube and the part of the external tube are not combined mutually so that the part of the external tube forms one overcoat sheath of the balloon configuration to be inflated, and
wherein, in the inner radial direction of the drain passageway configured in the internal tube, one part of the interior surface of the drain passageway and one part of the interior surface of the external tube are combined mutually;
wherein one end of the balloon configuration to be inflated starts at the distal end of the catheter body.

12. The balloon catheter configuration according to claim 11, wherein further comprising one stripping agent formed at the interface between the internal tube and the external tube and is configured at the first end part of the catheter body.

13. The balloon catheter configuration according to claim 11, wherein the part of the interior surface of the drain passageway is one rough surface.

14. The balloon catheter configuration according to claim 13, wherein the rough surface comprises one concave-convex surface, one spiral surface, or a combination of thereof.

15. The balloon catheter configuration according to claim 11, wherein the balloon catheter further comprises one infusion connector configured at the other end of the catheter body, relative to the first end part of the catheter body, wherein the infusion connector comprising one air inlet and one feeding pipe, the air inlet communicates with the longitudinal groove of the internal tube mutually; the feeding pipe and the drain passageway of the internal tube are in communication mutually; and the air inlet and the feeding pipe are not in communication with each other.

16. The balloon catheter configuration according to claim 11, wherein the diameter of the internal tube and the diameter of the external tube configured in the catheter body are reduced respectively toward the first end part of the catheter body.

17. The balloon catheter configuration according to claim 11, wherein the diameter of the internal tube and the diameter of the external tube configured in the catheter body are increased respectively toward the first end part of the catheter body.

18. The balloon catheter configuration according to claim 11, wherein the internal tube further comprises one separation structure configured in the drain passageway of the internal tube, the separation structure is configured connectedly to the interior surface of the drain passageway; and wherein the separation structure extends in the axial direction of the internal tube and shunts the flow volume configured inside the drain passageway of the internal tube.

19. The balloon catheter configuration according to claim 18, wherein one end of the separation structure is retracted into the internal tube, and the length of the internal tube in the axial direction is greater than the length of the separation structure in the axial direction.

20. The balloon catheter configuration according to claim 18, wherein the separation structure further comprising: one sheet-plate structure, one triangular-pyramid structure, one polygonal structure, one herringbone structure, one H-shaped structure, one cross structure, or a combination of thereof.
